(19) United States
(12) Patent Application Publication
(10) Pub. No.: US 2004/0096816 A1
(43) Pub. Date: May 20, 2004

(54) Method of indentifying an eventual modification of at least one biological parameter implementing young and aged living cells

(75) Inventors: Eric Perrier, Les Cotes d'Arey (FR); Francoise Pivard, Lyon (FR); Jean-Eric Branka, Chavanay (FR); Valerie Andre, Ampuis (FR)

Correspondence Address: MERCHANT & GOULD PC, P.O. BOX 2903, MINNEAPOLIS, MN, 55402-0903, US

(73) Assignee: COLETICA, Lyon (FR)

(21) Appl. No.: 10/365,894

(22) Filed: Feb. 12, 2003

(30) Foreign Application Priority Data
Nov. 19, 2002 (FR) 02 14492

(51) Int. Cl. C12Q 1/00; C12Q 1/68

(52) U.S. Cl. 435/4; 435/6

(57) ABSTRACT

An object of the invention is a method of identifying an eventual modification of at least one biological parameter.

The present invention relates essentially to a method of identifying an eventual modification of at least one biological parameter, comprising the compared proteomic and/or compared transcriptomic and/or compared genomic analysis:

a) of living young living cells,

b) of living aged living cells,

c) at least one of these two classes of cells being used in a three-dimensional tissue model,

enabling eventually identifying at least one biological parameter which is modified further to cell ageing.

The invention comprises the use of this process for the screening of active principles.

The present invention relates essentially to a method of identifying an eventual modification of at least one biological parameter, comprising the compared proteomic and/or compared transcriptomic and/or compared genomic analysis:

a) of young living cells,

b) of aged living cells,

c) at least one of these two classes of cells being used in a three-dimensional tissue model,

enabling eventually identifying at least one biological parameter which is modified following cell ageing.

The present invention further relates to a method of identifying at least one potentially active substance capable of reversing at least one biological parameter modified during ageing, or to provide an indication of the modification of at least one biological parameter modified during ageing.

The present invention further relates to the use of an active substance selected by such a method, for preparing at least one cosmetic and/or pharmaceutical composition.

The present invention further relates to a substance which is active in the field of cosmetics or of pharmacy and which is selected by such a method.

Cells which are called <<young>> cells are either cells which originate from biopsies from young donors, or cells which have not been multiplied very much, corresponding to a relatively low number of in vitro passages, or cells taken from biopsies not exposed very much to solar radiation (body, breast, abdomen, foreskin, etc . . . ), cells which are called <<aged>> being then either cells which originate from biopsies from aged donors, or cells which have undergone a significant number of in vitro passages, or cells which originate from biopsies taken in areas which are exposed to the sun (neck, hand, face, etc . . . ).

The analysis by various genomic and proteomic methods of the cell models thus made enables demonstrating potential therapeutic targets and selecting cosmetic or pharmaceutical active principles, in order to modulate the effects of ageing which are thus identified. The effectiveness of cosmetic or pharmaceutical formulations containing such active principles can also be evaluated on these cell models.

### STATE OF THE ART

Every cell function, including cell proliferation, differentiation and death, are controlled by numerous genes and cell signal pathways. However, the analysis of the results obtained in vitro on monolayer cell cultures, generally of fibroblasts or keratinocytes, which originate from healthy or pathological donors, or from cell lines, is not always in perfect appropriateness with those obtained on biopsies.

In fact, the regulation of the cell proliferation and of the metabolic syntheses is completely different between monolayer cell models and a biopsy or a three-dimensional cell model which is a very close representation of it. Similarly, in the three-dimensional multicellular models, mechanisms of intercellular regulations have been demonstrated. They are due either to the interconnection of the cell types, or to the presence of diffusable factors which enable for example the regulation of keratinocytes by the fibroblasts and vice versa (Saintigny G., Bonnard M., Damour O. and Colombel C. in Acta Derm Venerol (Stockh) (1993) 73: 175-180 and Lacroix M., Bovy T., Nusgens B. V. and Lapiere C. M. in Arch Dermatol Res (1995) 287: 659-664), or the differentiation of dendritic precursors into endothelial cells and into macrophages in more complex models of immunocompetent reconstructed skins (A. Black: Structure maturation et dynamique d'un modèle de peau reconstruite humaine (<<Structure maturation and dynamics of a model of human reconstructed skin>>); Thesis 82/2000 UCBL1, France).

Moreover, it is not always possible to find data on protein expression and syntheses as a function of the physiological or photo-induced ageing for example, or these data sometimes reveal to be contradictory to what can be observed in vivo due to the simplified model used in the experimentation.

The appearance of the technologies originating from proteomic and genomic, such as bi-dimensional electrophoreses, protein <<arrays>> and DNA <<arrays>>, do at present enable the detection of multiple parameters in a very sensitive way, and, consequently, the demonstration of variations in expression of genes or in protein syntheses from biological samples of small size.

Up to present, these technologies have been used on various types of biological samples such as human sera or culture media conditioned by monolayer human cells (Huang R. P., Huang R., Fan Y. and Lin Y., in Analytical Biochemistry (2001) 294 : 55-62) or in monolayer cultures of various cell types and, for example, of fibroblasts and of melanocytes (Gutsmann-Conrad A., Heydari A. R., You S. and Richardson A., in Exp. Cell Res. (1998) 241(2): 404-413). Likewise, experiments have been made on keratinocytes which originate from donors of varying ages cultivated in a monolayer (Compton C., Tong T., Trookman N., Zhao H. and Roy D,. in J. Invest. Dermatol. (1994)103(1): 127-133) or fibroblasts which originate from donors of varying ages cultivated in a monolayer (Reed M. J., Ferara N. S. and Vernon R. B., in Mechanisms of ageing and development (2001) 122(11): 1203-1220) or on fibroblasts which are aged in vitro (Nishio K., Inoue A., Qiao H. K., Mimura A., in Histochem Cell Biol (2001) 116 : 321-327).

Certain experimentations have also enabled the study of the modulator role of an ultraviolet stress on normal or malignant human melanocytes (cell line or melanocytes extracted from tumor tissue) which are cultivated in a monolayer (Valery C., Grob J. J. and Verrando P., in J. Invest. Dermatol. (2001) 117: 1471-1482).

In parallel to the development of analytical techniques, numerous models of epidermis (EP 0 789 074 A1 of loreal) or of reconstructed epithelia (Schmalz G. Schweikl H. and Hiller K. A. in Eur. J. Oral Sci. (2000) 108: 442-448), but also of reconstructed mucous membranes, of reconstructed skins or of pigmented and/or immunocompetent reconstructed skins, have been made (EP 0 296 78 of the CNRS). These models are very widely used today for the pharmaco-toxicological evaluations and the study of effectiveness of pharmaceutical and cosmetic ingredients. However, the methods of analysis employed are essentially techniques of histology which are combined with image analysis, analyses of metabolic syntheses and of their regulation by electrophoretic, Western-blot Northern-blot or RT-PCR analysis. The protein array techniques and DNA array techniques or combined determinations of cytokines in particular, have never been applied to these models using young and aged cells.

### AIMS OF THE INVENTION

A main aim of the invention is to unexpectedly solve the technical problem which consists in providing a study model of cell metabolism, which reflects the situation observed in vivo, when the cells have undergone an ageing.

An aim of the invention is to solve the novel technical problem which consists in providing a method of identifying an eventual modification of at least one biological parameter, characterized in that it comprises the compared proteomic and/or compared transcriptomic and/or compared genomic analysis

a) of young living cells,

b) of aged living cells,

c) at least one of these two classes of cells being used in a three-dimensional tissue model,

enabling eventually identifying at least one biological parameter which is modified further to cell ageing.

An aim of the present invention is to carry out the study of the genomic and protein profile of compared cell models. This comparison is made between, on the one hand, a model selected from:

1) reconstructed epithelia (including the reconstructed epidermis), pigmented and/or immunocompetent reconstructed epithelia, connective matrices (including chorions, including dermis), reconstructed skins or mucous membranes, pigmented and/or immunocompetent reconstructed skins or mucous membranes, pigmented and/or immunocompetent and/or endothelialised and/or macrophage-containing reconstructed skins or mucous membranes, biopsies,

and, on the other hand:

2) the models above, or the cells making up the various models described above, which are cultivated in a monolayer or in suspension,

the comparison being made between young cells and aged cells:

cells called <<young cells>>, i.e. cells which are extracted from biopsies from donors of a young age, or which are not very amplified in vitro or which are extracted from biopsies originating from areas of the body which are non-exposed to the sun (body, breast, abdomen, foreskin, . . . )

cells called <<aged cells>>, i.e. cells which are extracted from biopsies from donors of senior age, or which are much amplified in vitro or which are extracted from biopsies originating from areas of the body exposed to the sun (neck, face, hand . . . ).

This proteomic or genomic comparison enables defining targets of action so as to reverse or to provide an indication of at least one biological parameter modified during ageing, whether this ageing be physiological or whether it be photo-induced.

Another aim of the present invention is to provide a solution which enable the use of the above-described tissue models with the purpose of evaluating the effect upon the genomic or transcriptomic or proteomic profile of an active principle, in particular a cosmetic or pharmaceutical active principle.

Another aim of the present invention is to provide a solution which enable the use of above-described tissue models with the purpose of evaluating the effect on the genomic or proteomic profile of a formulation, in particular a cosmetic or pharmaceutical formulation, which contains or does not contain an active principle.

### SUMMARY OF THE INVENTION

The present invention enables solving the technical problems set forth above.

Within the context of this invention, by <<genomic study>>, the inventors mean the act of drawing up an inventory of the different genes which expressions are modified, in order to modify their expression.

By <<transcriptomic study>>, the inventors mean the act of drawing up an inventory of the different RNAs which expressions are modified, in order to modify their expression.

By <<proteomic study>>, the inventors mean the act of drawing up an inventory of the different proteins which expressions are modified, in order to modify their expression.

The invention consists mainly of providing a method of identifying an eventual modification of at least one biological parameter, characterized in that it comprises the compared proteomic and/or compared transcriptomic and/or compared genomic analysis:

a) of young living cells,

b) of aged living cells,

c) at least one of these two classes of cells being used in a three-dimensional tissue model,

enabling eventually identifying at least one biological parameter which is modified further to cell ageing.

<<Young cells>> are either cells which originate from biopsies from young donors, or cells which have not been multiplied very much, corresponding to a relatively small number of in vitro passages, or cells taken from biopsies which have not been exposed very much to solar radiation (body, breast, abdomen, foreskin, etc . . . ).

<<Aged cells>> are then either cells which originate from biopsies from aged donors, or cells which have undergone a significant number of in vitro passages or cells which originate from biopsies taken in areas which are exposed to the sun (neck, hand, face, etc . . . ).

By passage the inventors mean:

the act of amplification by trypsination.

The tissue cell model is defined as being a tissue model also called a three-dimensional model, which can be sown with living cells, notably with the aim of reconstituting the tissue of a living being, in particular the tissue model is defined as being able to be a connective matrix model, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, a mucous membrane model constituted of an epithelium and of a chorion, of a model of biopsies (or explants) maintained in survival, as well as the models in monolayer or in suspension making use of the cells present in the models described above.

Use in these models can be made of normal, healthy or pathological cells, or of cells which originate from cell-lines; these cells can be of human or animal origin.

According to a variant of this latter characteristic, the three-dimensional culture model of connective matrices (dermis or chorion), comprises a support sown with stromal cells in order to form reconstructed dermis or reconstructed chorions.

The three-dimensional epidermis or epithelium culture model comprises a support sown or not beforehand with stromal cells, in particular with fibroblasts, and then with epithelial cells and in particular keratinocytes in order to obtain reconstructed epithelia or epidermis.

The three-dimensional reconstructed skin or mucous membrane culture model comprises a matrix support (dermal or of chorion) sown with epithelial cells in order to obtain a reconstructed mucous membrane or keratinocytes in order to obtain a reconstructed skin.

According to a variant, the three-dimensional culture model used comprises a model in which at least one additional cell type has been incorporated, e.g. endothelial cells (EC) and/or lymphocytes and/or adipose cells and/or interstitial dendritic cells and/or skin appendices, such as body hair, hair, sebaceous cells.

Advantageously, pigmentary cells, immunocompetent cells (Langerhans cells), nerve cells . . . can be introduced to the epithelial part.

The various cell types (fibroblasts, keratinocytes, melanocytes) extracted are amplified separately but can be used separately or pooled from several donors for the reconstruction of the three-dimensional models as well as for the cultures in monolayer or in suspension.

According to a variant, the dendritic precursors (interstitial dendrite cells) can differ spontaneously and initiate at least two additional cell types such as endothelial cells and macrophages, when the dendritic precursors are cultivated in a three-dimensional environment comprising at least epithelial and stromal cells.

The tissue models defined above are used at the end of the culture in order to make genomic and/or transcriptomic and/or proteomic analysis, which enable in particular the selection, the identification and the characterization of potential targets so as to reverse or to provide an indication of at least one biological parameter modified during ageing, whether it be physiological or photo-induced.

The potential targets correspond to the biological parameters to be reversed or the modification of which is to be indicated, which are identified by virtue of the implementation of the invention.

After definitions of the targets, these same models and methods of detection can be used for the screening of cosmetic or pharmaceutical active principles, the demonstration of effectiveness of cosmetic or pharmaceutical formulations containing or not the actives.

Amongst the analytical techniques used, the following can be cited in particular:

for the analysis of the proteomic profile: bidimensional electrophoresis, and/or protein arrays and/or cytokine array, and/or combined ELISA,

for the analysis of the genomic profile: DNA arrays, and/or polymerase chain reaction multiplex (PCR-multiplex), and/or polymerase chain reaction (PCR), and/or real time polymerase chain reaction (real time PCR),

for the analysis of the transcriptomic profile: RNA arrays, cDNA arrays and/or reverse transcription polymerase chain reaction multiplex (RT-PCR-multiplex) and/or reverse transcription polymerase chain reaction (RT-PCR) and/or real time reverse transcription polymerase chain reaction (real time RT-PCR).

### DETAILED DESCRIPTION OF THE INVENTION

According to a first aspect, the invention relates to a method of identifying an eventual modification of at least one biological parameter, comprising the compared proteomic and/or compared transcriptomic and/or compared genomic analysis:

a) of young living cells,

b) of aged living cells,

c) at least one of these two classes of cells being used in a three-dimensional tissue model,

enabling eventually identifying at least one biological parameter which is modified further to cell ageing.

Advantageously, the young cells and the aged cells are both used in a three-dimensional tissue model.

Advantageously, said biological parameter, which is modified during the cell ageing, is defined by at least one difference between the metabolism of the young cells and the metabolism of the aged cells.

Advantageously, the young cells of step a) cited above are either cells which originate from biopsies from young donors, of an age advantageously of less than 40-45 years old, or cells which have not been multiplied very much, corresponding to a relatively small number of in vitro passages, or cells taken from biopsies which have not been exposed very much to solar radiation (e.g. the body, the breast, the abdomen, the foreskin).

Advantageously, the aged cells of step b) cited above are either cells which originate from biopsies from aged donors, of an age advantageously greater than 40-45 years old, or cells which have undergone a significant number of in vitro passages, or cells which originate from biopsies taken in areas which are exposed to the sun (e.g. the hand, the face, the neck, the nape).

Advantageously, the aged cells of step b) cited above are young cells which have been integrated in a three-dimensional tissue model comprising one or more cell types, artificially aged by a culture prolonged over a long period, advantageously greater than 1 month, more advantageously greater than 2 months.

Advantageously, said young cells or aged cells are cells from at least one human being or from at least one animal.

Advantageously, said study comprises at least one analysis selected from the following methods of analysis:

for the analysis of the proteomic profile: bidimensional electrophoresis, and/or protein arrays and/or cytokine array, and/or combined ELISA,

for the analysis of the genomic profile: DNA arrays, and/or polymerase chain reaction multiplex (PCR-multiplex), and/or polymerase chain reaction (PCR), and/or real time polymerase chain reaction (real time PCR),

the analysis of the transcriptomic profile: RNA arrays, cDNA arrays and/or reverse transcription polymerase chain reaction multiplex (RT-PCR-multiplex) and/or reverse transcription polymerase chain reaction (RT-PCR) and/or real time reverse transcription polymerase chain reaction (real time RT-PCR).

Advantageously, said tissue model is cultivated and/or preserved under conditions which maintain, at least partially, a cell metabolism.

Advantageously, said tissue model comprises at least fibroblasts or keratinocytes.

Advantageously, said model comprises normal, healthy or pathological cells, or cells which originate from cell-lines, preferably these cells are of human or animal origin.

Advantageously, said tissue model is selected from the following models: a connective matrix model, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, a mucous membrane model constituted of an epithelium and of a chorion.

Advantageously, said tissue model is a connective matrix tissue model: (dermis or of chorion) comprising a matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, polyethylene terephthalate (PET), a semi-permeable Anopore inorganic membrane, a cellulose acetate or cellulose ester (HATF) membrane, a semi-permeable Biopore-CM membrane, a semi-permeable polyester membrane, a membrane or a film of polyglycolic acid. In this group, the dermal models Skin²™ model ZK1100 and Dermagraft® and Transcyte® (Advanced Tissue Sciences), for example, are found;

a cell culture-treated plastic (formation of a dermal leaf: Michel M. et al in In Vitro Cell. Dev Biol.-Animal (1999) 35: 318-326);

a gel or a membrane based on hyaluronic acid (Hyalograft® 3D-Fidia advanced Biopolymers) and/or on collagen and/or on fibronectin and/or on fibrin; in this group, dermal model Vitrix® (Organogenesis), for example, is found;

a porous matrix, which is surfaced or non-surfaced, made from collagen being able to contain one or more glycosaminoglycans and/or eventually chitosan (EP 0 296 078 A1 of the CNRS, WO 01/911821 and WO 01/92322 of Coletica). In this group, the dermal model Mimederm® (Coletica), for example, is found.

These matrix supports comprise stromal cells, in particular fibroblasts.

Advantageously, said tissue model is an epidermis tissue model or epithelium tissue model comprising a matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, of polyethylene terephthalate (PET), a semi-permeable Anopore inorganic membrane, a cellulose acetate or cellulose ester (HATF) membrane, a semi-permeable Biopore-CM membrane, a semi-permeable polyester membrane; in this group, the reconstructed Epidermis and Epithelia models (Skinethic®) as well as the models EpiDerm®, EpiAirway®, EpiOccular® (Mattek Corporation), are found;

a film or a membrane based on hyaluronic acid and/or on collagen and/or on fibronectin and/or on fibrin. In this group, the models Laserskin® (Fidian advanced Biopolymers), Episkin® (L'Oreal), can in particular be cited.

These models are sown with stromal cells, in particular fibroblasts, and then with epithelial cells and in particular keratinocytes.

Advantageously, on the epithelial part, epithelial cells, pigmentary cells, immunocompetent cells, nerve cells, are introduced in addition, preferably, the immonocompetent cells are Langerhans cells.

Advantageously, said tissue model is a reconstructed skin or mucous membrane tissue model comprising a dermal or chorion matrix support preferably selected from:

an inert support selected from the group consisting of a semi-permeable synthetic membrane, in particular a semi-permeable nitrocellulose membrane, a semi-permeable nylon membrane, a teflon membrane or a teflon sponge, a semi-permeable membrane of polycarbonate or polyethylene, polypropylene, of polyethylene terephthalate (PET), a semi-permeable Anopore inorganic membrane, a cellulose acetate or cellulose ester (HATF) membrane, a semi-permeable Biopore-CM membrane, a semi-permeable polyester membrane, said inert support containing stromal cells, in particular fibroblasts,

a gel based on collagen and/or hyaluronic acid and/or fibronectin, and/or on fibrin comprising stromal cells in particular fibroblasts,

a porous matrix, which is surfaced or non-surfaced, made from collagen being able to contain one or more glycosaminoglycans and/or eventually chitosan, these porous matrices integrating stromal cells, in particular fibroblasts,

a de-epidermisized dermis or dead dermis, human or animal.

In this group, the models Mimeskin® (Coletica), Apligra® (Organogenesis), ATS-2000 (CellSystems® Biotechnologie Vertrieb), as well as Skin²™ (ZK1200-1300-2000 -Advanced Tissue Science), can in particular be cited.

Moreover, models do exist which are dedicated to tissue therapy which can also be the subject of such studies. The models Epidex™ (Modex Therapeutiques), Epibase® (Laboratoire Genevrier), Epicell™ (Genzyme), Autoderm™ and Transderm™ (Innogenetics), can be cited. The matrix support is then sown with epithelial cells in order to obtain a reconstructed mucous membrane or with keratinocytes in order to obtain a reconstructed skin.

Advantageously, said tissue model used comprises a model in which at least one additional cell type has been incorporated, preferably endothelial cells (EC) and/or immune cells such as lymphocytes, macrophages, dendritic cells and/or adipose cells and/or skin appendices, such as body hair, hair, sebaceous cells.

According to a second aspect, the invention relates to the use of a method as defined above for carrying out the screening of at least one potentially active substance capable of reversing at least one biological parameter modified during ageing as defined above.

Advantageously, the method for carrying out the screening of at least one potentially active substance capable of reversing at least one biological parameter modified during ageing as defined above comprises:

A/ placing said potentially active substance in contact with the aged cells as defined above, which are sown in a cell model or tissue model as defined above, for a period of time sufficient to enable said potentially active substance to act;

B/ young cells as defined above, which are sown in a cell model or tissue model as defined above;

C/ the proteomic analysis and/or transcriptomic analysis and/or genomic analysis, partial or complete, for making the study of the action of said substance on the cell metabolism of said aged cells;

D/ comparing the cell metabolism of said aged cells in the presence of the potentially active substance, with the metabolism of said aged cells or of the young cells without the presence of said substance, and;

E/ identifying the presence or the absence of activity of said potentially active substance, notably comprises identifying a positive or negative effect of said substance in order to provide an indication of the modification of the biological parameter identified as being modified during ageing.

According to another aspect, the invention relates to a method of identifying at least one potentially active substance capable of reversing at least one biological parameter modified during ageing comprising:

a) culturing young cells preferably as defined above, used as reference;

b) culturing aged cells preferably as defined above, having a biological parameter modified with respect to the cells called young cells, in the presence of at least one potentially active substance, for a period of time sufficient to enable said potentially active substance to eventually act on the cell metabolism of said cells, so as to recover the metabolism of the young cells;

c) the proteomic analysis and/or transcriptomic analysis and/or genomic analysis, partial or complete, preferably as defined above, of the aged cells which are cultivated in the presence or not of an eventually active substance;

d) comparing the analysis carried out in c) with the proteomic analysis and/or transcriptomic analysis and/or genomic analysis, partial or complete, preferably as defined above, of living young living cells, which are cultivated without the presence of said potentially active substance, as described in a);

e) following the comparison of the analyses carried out in c) and d), eventually identifying at least one active substance capable of reversing at least one biological parameter modified by the ageing.

According to another aspect, the invention relates to a method of identifying at least one potentially active substance capable of providing an indication of the modification of at least one biological parameter modified during ageing comprising:

a) placing said potentially active substance in contact with the cells called aged cells as defined above, which are sown in a tissue model as defined above, for a period of time sufficient to enable said potentially active substance to act;

b) the proteomic analysis and/or transcriptomic analysis and/or genomic analysis, partial or complete, preferably as defined above, of the aged cells, placed in contact with these substances;

c) comparing the analysis carried out in b) with the proteomic analysis and/or transcriptomic analysis and/or genomic analysis, partial or complete, preferably as defined above, of living cells which are cultivated without the presence of said potentially active substance;

d) following the comparison of the analyses carried out in c), eventually identifying at least one active substance capable of providing an indication of the modification of at least one biological parameter modified by the ageing.

According to another aspect, the invention relates to the use of an active substance selected by a method as defined above, for preparing at least one cosmetic and/or pharmaceutical composition.

According to another aspect, the invention relates to a substance which is active in the field of cosmetics or of pharmacy selected by a method defined above.

According to another aspect, the invention relates to an active substance capable of reversing a biological parameter which is identified as being modified during ageing, and/or of providing an indication of the modification thereof, this parameter having been identified by making compared studies made between cell models making use of young cells and cell models making use of aged cells, one at least of these models being a tissue model comprising at least either fibroblasts, or keratinocytes.

Other aims, characteristics and advantages of the invention will appear clearly in the light of the explanatory description which follows and which is made in reference to the Examples which are given simply as an illustration and which in no way limit the scope of the invention. The Examples make up an integral part of the present invention, and any characteristic which appears novel with respect to any state of the art is claimed as an integral part of the invention in its function and in its generality. In the Examples, all percentages are given by weight, the temperature is given in degrees Celsius, the pressure is atmospheric pressure, unless indications to the contrary.

### EXAMPLES

### Example 1

### Extraction and Culture of Cells Called <<Young or Aged>> Cells

The cells which are called <<young>> cells are either:

cells extracted from young donors, i.e. extracted from biopsies obtained from plastic surgery, preferably foreskin or abdominal or mammary or eventually gingival or vaginal, which are non-exposed to the sun,

cells extracted from young donors, e.g. donors aged less than 45 years old,

cells used in early passage, e.g. less than 10 for the fibroblasts, less than 6 for the melanocytes and less than 2 for the keratinocytes.

The cells which are called <<aged>> cells are either:

cells which are extracted from biopsies from aged donors and which are obtained from plastic surgery, preferably abdominal or mammary or eventually gingival or vaginal, which are non-exposed to the sun, from aged patients, e.g. from donors of more than 45 years old,

cells which are extracted from biopsies from donors of varying age, and which are obtained from plastic surgery of areas exposed to the sun (face, neck, hand)

cells used in late passage, e.g. greater than 10 for the fibroblasts, greater than 6 for the melanocytes and greater than 2 for the keratinocytes.

The cell types obtained can be fibroblasts extracted by the technique of explants or by enzymatic digestion, e.g. with collagenase, keratinocytes or melanocytes extracted after enzymatic dermo-epidermic dissociation, in particular dispase or thermolysin or trypsin-EDTA . . . .

After extraction, the fibroblasts are amplified in DMEM medium (Dulabecco's Modified Eagle's Medium)/Ham F12 glutamax 50/50 volume/volume, supplemented with 10% calf serum, with penicillin at a final concentration of 100 UI/milliliter, with gentamycin at a final concentration of 1 microgram/milliliter, with amphotericin B at a final concentration of 1 microgram/milliliter. The fibroblasts are amplified by trypsination as soon as a confluence of 90% is obtained.

After extraction, the keratinocytes are amplified in K-SFM medium (Keratinocyte Serum Free Medium-Invitrogen) containing extract of bovine pituitary gland supplemented with penicillin at a final concentration of 100 UI/milliliter, with gentamycin at a final concentration of 1 microgram/milliliter, with amphotericin B at a final concentration of 1 microgram/milliliter. The keratinocytes are amplified by trypsination as soon as a confluence of 90% is obtained.

After extraction, the melanocytes are amplified in MMK2 medium (Melanocyte Medium Kit-Sigma) supplemented with penicillin at a final concentration of 100 UI/milliliter, with gentamycin at a final concentration of 1 microgram/milliliter, with amphotericin B at a final concentration of 1 microgram/milliliter and with geneticin at the rate of 100 micrograms/milliliter for 3 days in order to eliminate the residual keratinocytes. The culture is then continued in the same medium except the geneticin. The melanocytes are amplified by trypsination as soon as a confluence of 90% is obtained.

### Example 2

### Preparation of Reconstructed Dermis Called <<Young and Aged>> Reconstructed Dermis, and Extraction of RNA, of DNA and of Proteins

500,000 fibroblasts from a pool of three young donors (less than 45 years old) and aged donors (greater than 45 years old) amplified as described in Example 1 are sown in dermal substrates made up of collagen which is cross-linked with diphenylphosphorylazide, in a DMEM-glutamax medium supplemented with 10% of calf serum , ascorbic-2-phosphate at a final concentration of 1 millimolar, EGF or epidermal growth factor at a final concentration of 10 nanogram/milliliter, penicillin at a final concentration of 100 UI/milliliter, amphotericin B at a final concentration of 1 microgram/milliliter for a period of 21 days.

At the end of experimentation, the reconstructed dermis are ground in liquid nitrogen with the aid of a biopulverizer. The grindings are taken up into Tri Reagent® (T9424 Sigma, St Louis USA) and then extracted with chloroform. After centrifugation at 12,000 g for 15 minutes at 4° C., the RNAs are found in the upper layer, the DNAs in the lower layer and the proteins at the interface.

### Example 3

### Preparation of Reconstructed Epidermis Called <<Young and Aged>> Reconstructed Epidermis, and Extraction of RNA, of DNA and of Proteins

4.10⁶ keratinocytes called <<young>> keratinocytes (donor of age of less than 35 years old) and <<aged>> keratinocytes (donor of age of greater than 55 years old) amplified as described in Example 1 until passage 1 (first amplification by trypsination) are sown in Boyden chamber-type inserts (membrane of porosity 0.4 μm and diameter 25 mm) sown beforehand with a nutrient under layer of fibroblasts, in a DMEM-Glutamax/Ham F-12 (ratio 3/1 v/v) culture medium supplemented with 10% of Hyclone II calf serum, ascorbic acid-2-phosphate at a final concentration of 1 millimolar, EGF or epidermal growth factor at a final concentration of 10 ng/mL, hydrocortisone at a final concentration of 0.4 micrograms/milliliter, umulin at a final concentration of 0.12 UI/milliliter, Isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture from 3 to 8 days.

The cultures of keratinocytes are then placed at the air-liquid interface for 12 to 18 days in the same culture medium used for the immersion culture, except the calf serum, the hydrocortisone, the isuprel, the triiodothyronine and the umulin.

At the end of experimentation, the reconstructed epidermis which are present in the inserts are scraped off, collected and then are taken up into Tri Reagent® (Sigma) and are extracted with chloroform. After centrifugation at 12,000 g for 15 minutes at 4° C., the RNAs are found in the upper layer, the DNAs in the lower layer and the proteins at the interface.

### Example 4

### Preparation of Reconstructed Gingival Mucous Membrane Epithelia Called <<Young and Aged>> Reconstructed Gingival Mucous Membrane Epithelia, and Extraction of RNA, of DNA and of Proteins

1 to 2.10⁶ epithelial cells of gingival mucous membrane called <<young>> epithelial cells (Passage 1, first amplification by trypsination) and <<aged>> epithelial cells (Passage 4, fourth amplification by trypsination), which are extracted as described in Example 1, are sown in Boyden chamber-type inserts (membrane of porosity 0.4 μm and diameter 10 mm), in a DMEM-Glutamax/Ham F-12 (ratio 3/1 v/v) culture medium supplemented with 10% of Hyclone II calf serum, ascorbic acid-2-phosphate at a final concentration of 1 millimolar, EGF or epidermal growth factor at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10-9 mole/litre, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture from 3 to 8 days.

The cultures of epithelial cells are then maintained in immersion for 12 to 18 days in the same culture medium as that used for the immersion culture, except the percentage of calf serum which is lowered by 10% to 1%.

At the end of experimentation, the reconstructed epithelia are taken up into Tri Reagent® (Sigma) and are then extracted with chloroform. After centrifugation at 12,000 g for 15 minutes at 4° C., the RNAs are found in the upper layer, the DNAs in the lower layer and the proteins at the interface.

### Example 5

### Three-dimensional Multicellular Model of Reconstructed Skins Called <<Young and Aged>> Reconstructed Skins and Extraction of RNA, of DNA and of Proteins

400,000 fibroblasts called <<young>> fibroblasts (pool of three donors of less than 35 years old) and <<aged>> fibroblasts (pool of three donors of more than 55 years old) are extracted and amplified until passage 5 (fifth amplification by trypsination) as described in Example 1 and are then sown on dermal substrates based on surfaced collagen sponges.

Briefly, the dermal substrates are prepared according to the following protocol:

Drying at 25° C. of a 0.75% collagen gel in order to form a film

Depositing the collagen film on a 0.75% collagen gel

Lyophilisation for 24 h and cross-linking with DPPA (diphenylphosphorylazide 50 μl/g of collagen in dimethylformamide solvent and then pH 8.9 borate buffer)

After rinsing with demineralized water, the surfaced dermal substrates are lyophilized once again.

The medium used for the culture of the fibroblasts is a DMEM-Glutamax medium supplemented with 10% of hyclone II calf serum, ascorbic acid-2-phosphate at a final concentration of 1 millimolar, EGF or Epidermal growth factor at a final concentration of 10 ng/mL, penicillin at a final concentration of 100 UI/milliliter, amphotericin B at a final concentration of 1 microgram/milliliter, and gentamycin at a final concentration of 20 micrograms/milliliter, and for a period of culture of 14 days.

Then, 400,000 keratinocytes called <<young>> keratinocytes (pool of three of less than 35 years old) and <<aged>> keratinocytes (pool of three donors of more than 55 years old) which are extracted and amplified until passage 2 (second amplification by trypsination) as described in Example 1 are sown on the dermal equivalents in a DMEM-Glutamax/Ham F-12 (ratio 3/1 v/v) culture medium supplemented with 10% of Hyclone II calf serum, ascorbic acid-2-phosphate at a final concentration of 1 millimolar, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture of 7 days.

The cultures are then placed at the air-liquid interface for 21 days in the same culture medium used for the immersion culture, except the calf serum, the hydrocortisone, the isuprel, the triiodothyronine and the umulin.

At the end of experimentation, the reconstructed skins are taken up into Tri Reagent® (Sigma), ground in liquid nitrogen with the aid of a biopulverizer and then extracted with choloroform. After centrifugation at 12,000 g for 15 minutes at 4° C., the RNAs are found in the upper layer, the DNAs in the lower layer and the proteins at the interface.

### Example 6

### Three-Dimensional Multicellular Model of Reconstructed Skin Called <<Young and Aged>> Models, Containing Populations of Langerhans Cells, Interstitial Dendritic Cells, Macrophages and Endothelial Cells, and then Extraction of RNA, of DNA and of Proteins

### Generation of the Undifferentiated and Immature Dendritic Cells which are Capable of Orientating Themselves Preferentially in the Differentiation Pathway of the Langerhans Cells

The peripheral circulating blood was collected taking a venous blood sample from one or more human donors, in vacutainers supplemented in usual anti-coagulant products such as lithium-heparin.

The separation of the CD14⁺ monocytes from this circulating blood can be done advantageously according to the protocol described by Geissmann et al. in J. EXP. MED. Vol 187, No 6, 16 March 1998, pages 961-966 published by The Rockefeller University Press, in the following manner:

after centrifugation on a Ficoll® gradient (sodium diatrizoate/polysucchrose density 1.077; Lymphoprep Abcys 1053980), the mononucleated cells of the circulating blood are recovered and indirectly labeled with an antibody cocktail (mainly anti-CD3, or anti-fCluster of Differentiation 3, anti-CD7, anti-CD19, anti-CD45RA, anti-CD56, or Cluster of Differentiation anti-IgE or anti-immunoglobulin E) coupled to magnetic beads.

after passage on a magnetic column, only the non-magnetically-labeled monocytes are eluted.

The CD14⁺ monocytes are recovered in the eluate in proceeding by any physical method de separation -well-known to the person skilled in the art and notably by sedimentation or centrifugation, and are eluted as such for the subsequent cultures.

The CD14⁺ monocytes are then put into culture, at the rate of about 1 million per milliliter, in an RPMI 1640 culture medium supplemented with 10% of decomplemented fcetal calf serum, and initially containing two cytokines, namely cytokine GM-CSF at the rate of 400 UI/mL and cytokine TGFβ1 at the rate of 10 ng/mL.

The culture is done at 37° C. in a moist atmosphere containing 5% of CO₂. The culture medium is initially supplemented with a third cytokine, namely cytokine IL-13 at the rate of 10 ng/mL. Before at the most 2 days of culture, the same culture medium is added but not containing the IL-13 until the 6th day of culture. On the 6th day, undifferentiated and immature dendritic cells are generated which are capable of orientating themselves preferentially in the differentiation pathway in Langerhans cells:

about 60 to 80% of the dendritic cells which are generated in vitro express the Langerin protein, and CCR6 which is the specific receptor of MIP-3α;

the dendritic cells which are generated in vitro are strongly chemo-attracted by MIP-3α, and this demonstrates the functionality of the receptor CCR6;

the dendritic cells which are generated in vitro are immature since they do not express the maturity labels CD83, DC-LAMP and CCR7.

### The Three-dimensional Model is then made According to the Protocol

2.10⁵ fibroblasts amplified from the passage 3 to the passage 10 (tenth amplification by trypsination), as described in Example 1, are sown on dermal substrates based on collagen-glycosaminoglycan-chitosan, in a DMEM-Glutamax culture medium supplemented with 10% of hyclone II calf serum, ascorbic acid-2-phosphate at a final concentration of 1 millimolar, EGF or Epidermal growth factor at a final concentration of 10 ng/mL, penicillin at a final concentration of 100 UI/milliliter, amphotericin B at a final concentration of 1 microgram/milliliter, and gentamycin at a final concentration of 20 micrograms/milliliter, and for a period of culture of 21 days. The culture is continued for a further week in the medium described above except the EGF.

Then, 2.10⁵ keratinocytes amplified from the passage 0 to the passage 2 (second amplification by trypsination) as described in Example 1, and 1 to 3.10⁵ undifferentiated dendrite cells which are generated in vitro are sown on the dermal equivalents in a DMEM-Glutamax/Ham F-12 (ratio 3/1 v/v) culture medium supplemented with 10% of Hyclone II calf serum, ascorbic acid-2-phosphate at a final concentration of 1 millimolar, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture of 7 days.

The cultures are then placed at the air-liquid interface for 21 days in the same culture medium used for the immersion culture, except the calf serum, the hydrocortisone, the isuprel, the triiodothyronine and the umulin.

Under these conditions, the Langerhans cells are localized in the epidermis, the interstitial dendritic cells, the macrophages and the endothelial cells in the dermis.

At the end of experimentation, the immunocompetent reconstructed skins are taken up into Tri Reagent® (Sigma), ground in liquid nitrogen with the aid of a biopulverizer and then extracted with chloroform. After centifugation at 12,000 g for 15 minutes at 4° C., the RNAs are found in the upper layer, the DNAs in the lower layer and the proteins at the interface.

### Example 7

### Purification of the RNA, DNA and the Proteins of the Cell Models Described Above

The RNAs are dissolved at the rate of 1 μg/pl of water after precipitation with 2-propanol, centrifugation 15 minutes at 12,000 g, washing the plug with ethanol 80%, drying, treatment with DNAases (AMBION) and reading the absorbance at 260/280 nm.

The DNAs are precipitated by centrifugation 2,000 g, 5 minutes at 4° C., after removal of the upper layer containing the RNAS, with ethanol. The supernatant is preserved and contains the protein fraction. The plug containing the DNAs is rinsed with centrifugation 2,000 g, 5 minutes at 4° C. with a 0.1M citrate buffer 10% ethanol, 75% ethanol and then dried under vacuum for 5-10 minutes and finally dissolved in 8 millimolar sodium hydroxide. Centrifuged 10 minutes at 12,000 g in order to remove the insolubles and to adjust the concentration of DNA to 0.3 μg/μl after reading the absorbance at 260 nm.

The proteins contained in the ethanolic extract (supernatant) are precipitated with isopropanol after centrifugation 12,000 g for 10 minutes at 4° C., rinsed with three washings by centrifugation 7,500 g for 5 minutes at 4° C. with 0.3M guanidine HCl in 95% ethanol. The plug is then dried under vacuum for 5-10 minutes and then dissolved in 1% SDS.

### Example 8

### Study of Effectiveness of an Anti-Age Complex on Reconstructed Skins Called <<Young and Aged>> Reconstructed Skins

Reconstructed skins were prepared according to Example 5. An anti-age complex made up of three actives, including an active Basaline® (modified malt protein, Coletica, Lyon) stimulating the reconstruction of the basal membrane, was added or not (non-treated control) to the immersion culture medium at a concentration of 2.25%, and this for a period of time of 14 days.

### Immunohistochemical Studies

After 14 days of culture, the <<control>> reconstructed skins and the <<treated>> reconstructed skins were frozen in liquid nitrogen and cryocuttings were made with the aid of a cryomicropart (cryostat, Microm 500M).

The presence of total laminins, of 5-laminin, of collagen IV and of collagen VII was traced by immunohistochemical techniques. For the laminins, it was NCL-LAMININ, for the collagen IV, it was NCL-COLL-IV and for the collagen VII, it was NCL-COLL-VII. The antibody used for the 5-laminin was kalinin/Laminin β3. All these antibodies were coupled to diaminobenzidine (DAB), used as tracing agent.

All the operations necessary for the evidencing of the molecules studied (antibody-molecules bond, rinsings, tracing . . . ) were carried out with the aid of a immunohistochemistry robot (Nexes, Ventana).

Slides of the cuttings of reconstructed skins were made for each antibody with the aid of an Axiophot microscope (Zeiss) (magnification×40) and enable evidencing an organized basal membrane of good quality.

The results obtained show that the models of reconstructed skins express all the labels followed throughout this study : the total laminins, laminin 5, type IV collagen and type VII collagen. The labeling is more intense in the case of the reconstructed skins treated with the anti-age complex. The histological analysis does not however enable showing a real difference between the young skins and the aged skins.

### <<Dot-Blot>> Experiments

In a first step, the collagens are extracted from the cutaneous reconstructions after digestion with pepsin (20 mg/g dry of sample) in 0.5N acetic acid medium for 48 h at 4° C.

After centrifugation for 30 minutes at 15,000 g, the type I collagen is removed by precipitation in 0.7M NaCl 2 hours at 4° C. and centrifugation 13,000 rpm.

The type IV collagen contained in the supernatant is precipitated in 1,2M NaCl for one night at 4° C. and collected by centrifugation 13,000 rpm.

The type VII collagen contained in the supernatant is precipitated with a TCA/Acetone/DTT mixture (Biorad) for two hours at 4° C. and 45 minutes at −20° C., collected by centrifugation 13,000 rpm for 30 minutes, and then rinsed in DTT/Acetone by centrifugation.

The samples containing the different types of collagen are taken up into TBS buffer and are then transferred onto a nitrocellulose membrane. After saturation with TBS buffer containing 3% bovine serum albumin for 30 minutes, the membrane is rinsed in TBS buffer and then incubated 1 hour at ambient temperature and with agitation in the solution containing the primary antibody. After rinsing in TBS buffer containing 0.05% of Tween® 20 (ICI), the membrane is incubated in the second antibody for 1 hour at ambient temperature and with agitation. The signal is then amplified by using an amplification and tracing kit Opti-4CN substrate kit (Bio-rad) by following the recommendations of the supplier. The quantification of the bands by image analysis does not enable showing a significant effect of the anti-age active upon the synthesis of collagen IV and VII (problem of sensitivity).

### Real Time Quantitative PCR Experiments

The quantification of the mRNAs encoding actin, collagen IV and collagen VII was carried out in the models which were reconstructed by a real time quantitative PCR technique. For this, primers enabling the amplification of specific fragments of the collagens IV (231 pairs of bases) and VII (763 pairs of bases) and primers of actin sequences (541 pairs of bases) were used.

| | |
|---|---|
| Sense | 5′-GTACTGCAACCCTGGTGATGTCTGC-3′ |
| collagen IV | |
| Antisense | 5′-GAATATCCGATCCACAAACTCCGCC-3′ |
| collagen IV | |
| Sense | 5′-GCCACAGGATACAGGGTTTC-3′ |
| collagen VII | |
| Antisense | 5′-CACCACACGTAGTTCAATGC-3′ |
| collagen VII | |
| Sense ACTIN | GTGGGGCGCCCCAGGCACCA |
| Sense ACTIN | CTCCTTAATGTCACGCACGATTTC |

The extraction of the RNAs from the treated and non-treated reconstructed skins was carried out by following the protocol described in Example 7.

The RT-PCR reactions (Reverse Transcription Polymerase Chain reactions) are carried out by real time RT-PCR with the aid of an <<Opticon>> system (MJ Research.).

The reaction mixture (50 μl) introduced into the receptacles is the following, for each sample:

10 μl of RNA at the concentration of 5 ng/μl.

The primers of the various labels used

Reaction mixture (Qiagen) containing the reverse transcription enzyme and the enzyme DNA polymerase, the labeling agent SYBR Green I (fluorophore inserting itself in the DNA double strand during elongation step) and MgCl2.

The conditions of RT-PCR are the following:

Reverse transcription: 30 min at 50° C.,

PCR reactions: [15 sec at 94° C., 30 sec at 56° C. and 30 sec at 72° C.], 50 cycles.

The absence of contamination and the purity of the products amplified are verified via the melting curves of the amplified products of PCR. The products presenting a double peak or an abnormal melting temperature are eliminated.

### Analysis and Method of Calculation

The incorporation of fluorescence in the amplified DNA was measured continuously during the cycles of PCR. This system enables obtaining fluorescence measurement curves as a function of the number of PCR cycles, and thus to evaluate a relative quantity of amplified DNA.

In order to take account of the cell population present in the reconstructed skins, all the results were attributed to the signal <<actin>>, used as <<housekeeping gene>>.

Depending on the experimentation, the measurement threshold of the C(T) (=threshold cycle) is fixed for T which is between 0.05 and 0.01 and then an arbitrary unit of measurement is calculated for each gene according to the formula:

Sgene <<x>>=10⁷×(1/2)^{c(T)gene<<x>>}

C(T)gene <<x>> signifying measurement threshold of the C(T) (Threshold cycle) of the gene <<x >>.

The values of the genes of interest are attributed to the signal <<actin>> by calculation of the ratio:

R=Sgene<<x>>/Sactin.

These ratios are compared between the treated and non-treated samples. The results obtained demonstrate that the anti-age complex significantly increases the expression of the mRNAs encoding the type IV collagen (+65% p<0.05), type VII collagen (+63% p<0.05) in the aged reconstructed skin model.

### Example 9

### Analysis by DNA Array of Monolayers, Reconstructed Dermis and Reconstructed Skins, Comparison Between Models Called <<Young and Aged>> Models

The same cell stocks, at the same passage, are used in order to make the three cell models.

Fibroblasts (pool of three donors of ages of less than 35 years and greater than 55 years old) extracted as defined in Example 1 were cultivated in monolayer to confluence in DMEM/Ham F12 glutamax 50/50 v/v medium, supplemented with 10% of calf serum, penicillin at a final concentration of 100 UI/milliliter, gentamycin at a final concentration of 20 microgram/milliliter, amphotericin B at a final concentration of 1 microgram/milliliter. The mats are collected in Tri Reagent® (Sigma).

The reconstructed dermis called <<young and aged>> reconstructed dermis are prepared by the sowing of surfaced collagen matrices which are cross-linked with diphenylphosphoryl azide with 400,000 fibroblasts (which originate from a pool of at least three donors of ages of less than 35 years old and greater than 55 years old, which are extracted and amplified separately according to the protocol described in Example 1). The reconstructed dermis are cultivated for 15 days in DMEM-glutamax medium supplemented with 10% of calf serum, ascorbic acid at a final concentration of 1 millimolar, EGF or epidermal growth factor at a final concentration of 10 nanogram/milliliter, penicillin at a final concentration of 100 UI/milliliter, amphotericin B at a final concentration of 1 microgram/milliliter, gentamycin at a final concentration of 20 microgram/milliliter. The reconstructed dermis are collected in Reagent® (Sigma).

The reconstructed skins called <<young and aged reconstructed skins>> are prepared by sowing of surfaced collagen matrices which are cross-linked with diphenylphosphoryl azide with 400,000 fibroblasts (which originate from a pool of at least three donors of ages less than 35 years and greater than 55 years old, which are extracted and amplified separately according to the protocol described in Example 1). The reconstructed dermis thus prepared are cultivated for 15 days in DMEM-glutamax medium supplemented with 10% of calf serum, ascorbic acid at a final concentration of 1 millimolar, EGF or epidermal growth factor at a final concentration of 10 nanogram/milliliter, penicillin at a final concentration of 100 UI/milliliter, amphotericin B at a final concentration of 1 microgram/milliliter, gentamycin at a final concentration of 20 microgram/milliliter. The keratinocytes (which originate from the same pools of donors and which are extracted and amplified separately according to the protocol described in Example 1) are sown at the rate of 400,000 per reconstructed dermis. The culture is continued for one week in proliferation medium made up of DMEM-Glutamax/Ham F-12 (ratio 3/1 v/v) supplemented with 10% of Hyclone II calf medium, ascorbic acid-2-phosphate at a final concentration of 1 millimolar, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter. The reconstructed skins are then emerged and then cultivated for two additional weeks in the same medium except the calf serum, the hydrocortisone, the isuprel, the triiodothyronine and the umulin. The reconstructed skins are collected in Tri Reagent® (Sigma).

### cDNA Array

Briefly, the RNAs of the samples are extracted (after grinding in liquid nitrogen with the aid of a Biopulverizer for the three-dimensional models) and purified according to the protocol of the supplier of Tri Reagent® (total elimination of the DNA).

The purified RNAs are analyzed qualitatively and quantitatively.

The following step was the purification of the pools of messenger RNAs (mRNAs) by hybridization of the poly(A) ends of the mRNAs with biotinylated oligo(dT) primers and selective capture on streptavidine beads, according to the Atlas Pure (Clontech) protocol. The DNA probes which are multiply labeled with 33p were made by reverse transcription of the mRNAs linked onto beads of poly(dT), with the aid of a pool of primers which are specific of the sequences which are immobilized on the arrays, in the presence of [(α³³P]-dATP. The labeled probes were purified by exclusion column chromatography, the quality and the equivalence of the labeled probes were evaluated by liquid scintillation counting.

The Custom ATLAS membranes were pre-treated and then the cDNAs which are immobilized on each membrane were hybridized (68° C., one night) with the corresponding labeled probes; the filters were then washed before analysis.

Analysis by autoradiography and quantification of the radioactivity of the spots with the aid of a Cyclone Phosphorimager (Packard instrument; 3 hours and then 72 hours of acquisition) and of the QuantArray, Packard, software.

Identification of the genes of interest varying between the different experimental conditions: young donors versus aged donors. The results are expressed in percentage of variation between the aged model and the young model, in monolayer condition, monocellular three-dimensional condition and multicellular three-dimensional condition.

If one is interested more particularly with the proteins of the extra-cellular matrix, it was thus possible to demonstrate for example that the quantity of RNA encoding the precursor of the fibronectin was increased in the monolayers (×2.1) and the reconstructed dermis (×1.8) called <<aged reconstructed dermis>> in comparison to the models called <<young>>. In contrast, in the reconstructed skin model called <<aged reconstructed skin model>>, the amount of RNA is clearly decreased (1.75) with respect to the reconstructed skin model called -<<young reconstructed skin model>>. The results obtained in the reconstructed skin model corroborate entirely the results obtained by immunohistochemical analyses of samples of skin from donors of varying ages. The results obtained on the monolayers which show an increase of the RNAs of precursors of fibronectin, are in agreement with results of studies obtained by analysis of the mRNAs which equally show an increase of the messenger as a function of the age of the donor but also of the number of passages of the fibroblasts cultivated in monolayer. These results illustrate entirely the fact that the results are dependent upon the cell models used and that the monolayer models, or unicellular three-dimensional models are not totally predictive since they do not take into account the interactions between the different cell types.

Moreover, it was possible to be demonstrated that numerous other genes have a different expression between the fibroblast monolayers, the reconstructed dermis and the reconstructed skins and that the levels of expression of these genes are not always equivalent in these three different models. The following results can be cited for example (results are expressed by the aged/young ratio in percentage):

TABLE I

| | Fibroblasts in | Reconstructed | Reconstructed |
|---|---|---|---|
| Gene | monolayer | dermis | skins |
| cytokeratin 1 | 118 | 178 | 85 |
| Alpha-2-PRAP et LRPAP1 | 292 | 95 | 81 |
| Bullous pemphigoid antigen 1 | nd | Nd | 232 |
| Cartilage specific proteoglycan core | 129 | 155 | 53 |
| protein, aggrecan core protein | | | |
| precursor, CS proteoglycan core | | | |
| protein 1 | | | |
| CD44 antigen precursor, ECMIII, | 77 | 33 | 89 |
| LHR, hyaluronate receptor, HS | | | |
| proteoglycan, epican | | | |
| CD59 glycoprotein precursor, | 95 | 40 | 83 |
| membrane attack c< | | | |
| CD9 antigen, P24, leucocyte | 40 | 124 | 102 |
| antigen MIC3, motility related | | | |
| protein | | | |
| Procollagen 3 alpha 1 subunit | 46 | 120 | 46 |
| precursor | | | |
| Collagen 6 alpha 1 subunit | 285 | 92 | 64 |
| Collagen 6 alpha 2 subunit | 154 | 99 | 80 |
| Collagen 16 alpha 1 subunit | 139 | 48 | 79 |
| precursor | | | |
| Elastin precursor | 36 | Nd | nd |
| Endothelial plasminogen activtor | 95 | 252 | 87 |
| inhibitor 1 precursor | | | |
| Fibronectin precursor | 209 | 177 | 57 |
| Hyaluronan synthase 2 | 109 | 282 | 189 |
| Involucrin | nd | Nd | 260 |
| LRP1, alpha 2 macroglobulin | 226 | 187 | 65 |
| receptor, apolipoprotein E Receptor, | | | |
| CD91 antigen | | | |
| Lumican precursor, keratan sulfate | 95 | 21 | 109 |
| proteoglycan, LDC | | | |
| MMP11, stromelysin 3 | 189 | 217 | 63 |
| MMP16 precursor, membrane type | 129 | 201 | 135 |
| matrix metalloproteinase 3, MMP- | | | |
| X2 | | | |
| MMP3, stromelysin 1 precursor | 7 | 117 | 143 |
| TIMP1, EPA, fibroblast collagenase | 40 | 49 | 74 |
| inhibitor | | | |
| TIMP3, mitogen inducible gene 5 | 49 | 210 | 173 |
| PAI-2, monocyte ARG-serpin, | nd | 88 | 333 |
| urokinase inhibitor | | | |
| SPARC, osteonectin, BM40 | 65 | 135 | 50 |
| Bone derived growth factor 1 | 213 | 141 | 93 |
| Cystein rich fibroblast growth factor | 101 | 124 | 49 |
| receptor, Golgi membrane | | | |
| sialoglycoprotein MG160 | | | |
| TIS11B protein, BRF1, EGF | 237 | 165 | 78 |
| response factor 1 | | | |
| Glia-derived neurite promoting | 49 | 137 | 53 |
| factor | | | |
| GCP2, neutrophil activating peptide | nd | Nd | 562 |
| ENA-178 | | | |
| Growth inhibitory factor, | 66 | 148 | 161 |
| metallothionein III | | | |
| Insuline like growth factor binding | 131 | 291 | 91 |
| protein 3 precursor | | | |
| IL1 alpha precursor, hematopoietin | nd | Nd | 161 |
| 1 | | | |
| IL1 receptor antagonist protein | nd | 88 | 156 |
| precursor | | | |
| IL1 receptor type II precursor | nd | Nd | 512 |
| IL12 beta subunit precursor | 137 | 173 | 81 |
| IL3 precursor, MCS factor, MCGF, | 114 | 180 | 75 |
| P-cell stimulating factor, | | | |
| hematopoietic growth factor | | | |
| IL6 precursor, BSF2, IFN beta 2, | 86 | 293 | 84 |
| hybridoma growth factor | | | |
| IL8 precursor, MDNCF, NAP1, | nd | 121 | 737 |
| LYNAP, protein 3-10C | | | |
| KGF, FGF7 | 65 | 150 | 50 |
| MIF, GIF | 35 | 193 | 121 |
| Calgranulin, leucocyte L1 heavy | nd | nd | 1981 |
| chain, S100A9, MRP14, | | | |
| Calgranulin A, MRP8, , leucocyte L1 | nd | nd | 4593 |
| light chain, S100A8 | | | |
| MCP1, MCAF, SCYA2 | 94 | 174 | 289 |
| Paxillin | 154 | 160 | 71 |
| Placenta growth factor 1 et 2 | 90 | 188 | 388 |
| PDGFA subunit precursor | nd | nd | 397 |
| PDGF receptor alpha subunit | 49 | 118 | 50 |
| Pleiotrophin precursor, osteoblast | 84 | 143 | 49 |
| specific factor 1, , HBNF1, HB-GAM, | | | |
| HB-GF8 | | | |
| Related to receptor tyrosine kinase | 122 | 159 | 80 |
| Rho-related GTP-binding protein | 132 | 88 | 188 |
| Thymosin beta 10, PTMB10 | 44 | 139 | 138 |
| TNF inducible protein, hyaluronate | nd | nd | 482 |
| binding protein | | | |
| VEGF B precursor, VEGF related | 163 | 98 | 106 |
| factor 186 | | | |
| VEGF precursor, vascular | 105 | 230 | 114 |
| permeability factor | | | |
| Zyxin 2 | 152 | 75 | 114 |
| HSP-90, HSP84, HSPCB | 97 | 40 | 107 |
| HSPB3, HSP17, HSPL27 | 97 | 168 | 95 |
| Cytosolic SOD1 | 88 | 47 | 120 |
| SOD2 | 88 | 47 | 120 |
| Metallothionein IH, MT0, MT1I, | 53 | 127 | 150 |
| MT2, MT1L, MT1R | | | |

The methods of the invention therefore enable a better comprehension of the syntheses of various molecules during ageing of the skin and can enable the screening of active principles aiming to provide an indication of or to limit the modifications observed during ageing.

### Example 10

### Analysis by Northern Blot of the mRNAs Encoding the Fibronectin in a Model of Reconstructed Skins Called <<Young and Aged Reconstructed Skins>>

The young and aged reconstructed skins are prepared by sowing of Collagen-GAG-Chitosan matrices with 400,000 fibroblasts (which originate from a pool of at least three donors of ages of less than 35 years and of greater than 55 years old, and which are extracted and amplified separately according to the protocol described in Example 1) and culture for 15 days in DMEM-glutamax medium supplemented with 10% of calf serum, ascorbic acid at a final concentration of 1 millimolar, EGF or epidermal growth factor at a final concentration of 10 nanogram/milliliter, penicillin at a final concentration of 100 UI/milliliter, amphotericin B at a final concentration of 1 microgram/milliliter, gentamycin at a final concentration of 20 microgram/milliliter. The keratinocytes (which originate from the same pool of donors and which are extracted and amplified separately according to the protocol described in Example 1) are sown at the rate of 400,000 per reconstructed dermis. The culture is continued for one week in proliferation medium made up of DMEM-Glutamax/Ham F-12 (ratio 3/1 v/v) supplemented with 10% of Hyclone II calf serum, ascorbic acid-2-phosphate at a final concentration of 1 millimolar, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter. The reconstructed skins are then emerged and then cultivated for two additional weeks in the same medium except the calf serum, the hydrocortisone, the isuprel, the triiodothyronine and the umulin. The samples are collected in Tri Reagent® (Sigma).

The total RNAs are extracted according to the protocol described in Example 7. After the quantification of the RNAs by densitometry at 260 nm, the solutions are adjusted to 1 μg/μl of RNA. 3 to 10 μg of each sample were then disposed on a gel of agarose/formaldehyde for separation. The RNAs were then transferred by capillarity on membranes of Hybond-N nylon (Amersham) for one night. The RNAs were covalently coupled to the nylon by heating at 80° C. for 90 minutes. The membranes were then prehybridized for 20 minutes at 68° C. in 8 ml of ExpressHyb (Clontech) at 0.1 mg/ml and hybridized for one night at 68° C. in the presence of labeled probes. At the end of the period of incubation, the membranes were washed 4 times 30 minutes at 68° C. with a twice-concentrated solution of SSC containing. 1% of SDS and then once at 68° C. with a 0.1-times concentrated solution of SSC containing 0.5% of SDS and finally a last time with a twice-concentrated solution of SSC. The direct counting of the radioactivity of the spots with the aid of a phosphoimager (Packard Instruments) enables the quantification of the RNAs. The results are expressed in percentage expression with respect to the actin signal. The quantitative analysis by Northern Blot of the mRNAs encoding the fibronectin enabled showing a decrease (factor 2) of the mRNAs in the models of reconstructed skins called <<aged>> reconstructed skins compared to the reconstructed skins called <<young>> reconstructed skins.

### Example 11

### Analysis by Western Blot of the Type VII Collagen in a Model of Reconstructed Skins Called <<Young and Aged>> Reconstructed Skins

The reconstructed dermis called <<young and aged>> reconstructed dermis are prepared by sowing of Collagen-GAG-Chitosan matrices with 400,000 <<young>> in P2 (passage 2, second amplification by trypsination) and <<aged>> in P12 (passage 12, twelfth amplification by trypsination), (extracted and amplified separately according to the protocol described in Example 1) and cultivated for 15 days in DMEM-glutamax medium supplemented with 10% of calf serum, ascorbic acid-2-phosphate at a final concentration of 1 millimolar, EGF or epidermal growth factor at a final concentration of 10 nanogram/milliliter, penicillin at a final concentration of 100 UI/milliliter, amphotericin B at a final concentration of 1 microgram/milliliter and gentamycin at a final concentration of 20 micrograms/milliliter. The keratinocytes called <<young>> in P1 (passage 1, first amplification by trypsination) and <<aged>> in P3 are extracted and amplified separately according to the protocol described in Example 1, and are then sown at the rate of 400,000 per reconstructed dermis. The culture is continued for one week in proliferation medium made up of DMEM-Glutamax/Ham F-12 (ratio 3/1 v/v) supplemented with 10% of Hyclone II calf serum, ascorbic acid-2-phosphate at a final concentration of 1 millimolar, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter. The reconstructed skins are then emerged and then cultivated for two additional weeks in the same medium except the calf serum, the hydrocortisone, the isuprel, the triiodothyronine and the umulin.

The type VII collagen is extracted according to the protocol described in Example 8 and is dissolved in electrophoresis buffer (0.5M Tris-HCl, pH 6.8, 10% glycerol, 2% SDS, 5% mercaptoethanol). The samples are heated at 95° C. for 5 minutes. The fractioning by size is carried out on 5% SDS-PAGE. The transfer is made on a polyvinylidene fluoride membrane. A control of purified collagen VII is made in parallel to the samples extracted, as well as a standard of molecular mass.

After transfer, the membranes are incubated for one hour in the blockage buffer (15 nM NaCl, 20 mM Tris-HCl, 0.5% Tween 20, 3% BSA, pH 7.4). The first anti-type VII collagen antibody (polyclonal rabbit 1/1000) is added in a 1% solution of PBS/BSA. After 2 hours at ambient temperature, the membranes are rinsed with the blockage buffer and are incubated with the second labeled antibody (HRP (horseradish peroxidase) conjugated anti-rabbit IgG for one hour at ambient temperature. After rinsing in PBS, the membranes are developed with a solution of 3,3′-diaminobenzidine tetrahydrochloride. In the case of weak labeling, use can be made of the amplification kit (Biorad) and then of the tracing kit Opti-4CN Substrate kit (Biorad).

After tracing, the membrane is rinsed a few minutes with water and then dried between absorbent paper.

The analysis of intensity of the bands by image analysis enables demonstrating a significant decrease in the content of type VII collagen of the extracts of the various aged samples.

### Example 12

### Analysis by DNA Array of Keratinocytes in Monolayer, in Comparison with Reconstructed Epidermis, Models Called <<Young and Aged>>

The same cell stocks, at the same passage, are used in order to make the three cell models.

Keratinocytes (pool of three donors of ages of less than 35 years and of greater than 55 years old) extracted as defined in Example 1 were cultivated in monolayer to confluence in K-SFM (Gibco) medium, penicillin at a final concentration of 100 UI/milliliter, gentamycin at a final concentration of 20 microgram/milliliter, amphotericin B at a final concentration of 1 microgram/milliliter. The mats are collected in Tri Reagent® (Sigma).

Reconstructed epidermis are prepared at the rate of 4.10⁶ keratinocytes called <<young>> (pool of three donors of ages of less than 35 years and of greater than 55 years old) amplified as described in Example 1 until passage 1 (first amplification by trypsination) are sown in Boyden chamber-type inserts (membrane of porosity 0.4 μm and diameter 25 mm) sown beforehand with a nutrient under layer of fibroblasts, in a DMEM-Glutamax/Ham F-12 (ratio 3/1 v/v) culture medium supplemented with 10% of Hyclone II calf serum, ascorbic acid-2-phosphate at a final concentration of 1 millimolar, EGF or epidermal growth factor at a final concentration of 10 ng/mL, hydrocortisone at a final concentration of 0.4 micrograms/milliliter, umulin at a final concentration of 0.12 UI/milliliter, Isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture from 3 to 8 days.

The cultures of keratinocytes are then placed at the air-liquid interface for 12 to 18 days in the same culture medium used for the immersion culture, except the calf serum, the hydrocortisone, the isuprel, the triiodothyronine and the umulin.

At the end of experimentation, the monolayers of keratinocytes and the reconstructed epidermis which are present in the inserts are scraped off, collected and then are taken up into Tri Reagent® (Sigma) and extracted with chloroform. After centrifugation at 12,000 g for 15 minutes at 4° C., the RNAs are found in the upper layer, the DNAs in the lower layer and the proteins at the interface.

The cDNA arrays are made as described in Example 9. The results obtained are the following (RE: relative unit of expression):

TABLE II

| Keratinocytes in monolayer | Young | Aged | A/Y |
|---|---|---|---|
| Name of the protein/gene | RE | RE | % |
| antichymotrypsin-alpha-1 (AACT; ACT) | 3.0 | 10.5 | 351 |
| cytokeratin 18 (CYK18; CK18) type I | 7.4 | 3.0 | 41 |
| cytoskeletal 18 keratin (KRT18; K18) | | | |
| S100 calcium-binding protein A4; placental | 6.5 | 17.8 | 273 |
| calcium-binding protein; calvasculin; | | | |
| MTS1 protein | | | |
| S100 calcium-binding protein A7; psoriasin | 10.3 | 3.1 | 30 |
| tropomyosin, skeletal muscle, alpha subunit | 9.0 | 27.5 | 305 |
| vimentin (VIM) | 20.2 | 41.0 | 203 |
| fibronectin (FN) | 18.3 | 53.4 | 291 |
| plasminogen activator inhibitor, type 1, | 10.7 | 32.4 | 302 |
| endothelial (PAI1; PLANH1) | | | |
| syndecan-4; amphiglycan; ryodocan core protein | 11.3 | 22.7 | 200 |
| tissue inhibitor of metalloproteinase 1 (TIMP1); | 3.7 | 9.1 | 247 |
| erythroid potentiating activity (EPA); fibroblast | | | |
| collagenase inhibitor | | | |
| steroid 5-alpha reductase 1 (SRD5A1); 3-oxo-5- | 3.8 | 8.0 | 212 |
| alpha steroid 4 dehydrogenase 1 | | | |
| bone-derived growth factor 1 (BPGF1) | 4.8 | 12.7 | 265 |
| cation-independent mannose-6-phosphate receptor | 3.8 | 8.2 | 218 |
| (CI man-6-P receptor; CI-MPR); insulin-like | | | |
| growth factor II receptor (IGFR II) | | | |
| transcription factor SREB1; sterol regulatory | 6.9 | 3.3 | 49 |
| element-binding transcription factor 1; SREBP1 | | | |
| vascular endothelial growth factor (VEGF); | 6.7 | 3.0 | 45 |
| vascular permeability factor (VPF) | | | |
| calmodulin-like skin protein (CLSP) | 24.8 | 6.1 | 25 |

TABLE III

| Reconstructed epidermis | Young | Aged | A/Y |
|---|---|---|---|
| Name of the protein/gene | RE | RE | % |
| antichymotrypsin-alpha-1 (AACT; ACT) | 106.4 | 37.3 | 35 |
| calcium-binding protein p22; calcium-binding | 3.0 | 7.2 | 238 |
| protein CHP | | | |
| corneodesmosin (CDSN); S protein | 3.0 | 6.8 | 225 |
| cytokeratin 10 (K10); type I cytoskeletal 10 | 28.6 | 82.5 | 289 |
| keratin | | | |
| cytokeratin 19 (K19; CK19); type I cytoskeletal | 46.1 | 3.0 | 7 |
| 19 keratin | | | |
| cytokeratin 6B (CK 6B; KRT6B; K6B); type II | 16.8 | 33.9 | 202 |
| cytoskeletal 6B keratin | | | |
| cytokeratin 7 (K7; CK7); type II cytoskeletal 7 | 131.3 | 9.7 | 7 |
| keratin (KRT7) | | | |
| desmocollin 2A/2B (DSC2); desmosomal | 3.0 | 15.7 | 524 |
| glycoprotein II/III | | | |
| desmoglein 1 (DSG1); desmosomal | 3.0 | 16.1 | 537 |
| glycoprotein 1 (DG1) | | | |
| desmoglein 3 (DSG3); 130-kDa pemphigus | 16.2 | 35.5 | 219 |
| vulgaris antigen (PVA) | | | |
| desmoplakin I & II (DSP; DPI & DPII) | 23.0 | 53.7 | 233 |
| integrin alpha 6 (ITGA6); VLA6; CD49F antigen | 7.4 | 15.9 | 215 |
| ribonuclease/angiogenin inhibitor (RAI; RNH); | 5.1 | 14.4 | 284 |
| placental ribonuclease inhibitor | | | |
| tropomyosin, skeletal muscle, alpha subunit | 21.1 | 8.0 | 38 |
| bone proteoglycan II (PGS2); decorin (DCN) | 5.0 | 10.7 | 213 |
| cell matrix adhesion regulator (CMAR; CAR) | 8.7 | 3.1 | 35 |
| collagen 1 alpha 1 subunit (COL1A1) | 6.0 | 3.0 | 50 |
| fibronectin (FN) | 12.4 | 5.3 | 43 |
| hyaluronan synthase 3 (HAS-3) | 3.0 | 6.8 | 228 |
| laminin beta 2 subunit (laminin B2; LAMB2); S- | 7.0 | 27.0 | 387 |
| laminin | | | |
| matrix metalloproteinase 1 (MMP1); interstitial | 3.0 | 52.2 | 1739 |
| collagenase (CLG); fibroblast collagenase | | | |
| matrix metalloproteinase 11 (MMP11); | 8.9 | 23.7 | 266 |
| stromelysin 3 | | | |
| matrix metalloproteinase 2 (MMP2); 72-kDa | 3.0 | 9.4 | 314 |
| gelatinase A; 72-kDa type IV collagenase | | | |
| (CLG4A); TBE-1 | | | |
| matrix metalloproteinase 3 (MMP3); stromelysin | 8.1 | 82.6 | 1023 |
| 1 (STMY1; SL1); transin 1 | | | |
| matrix metalloproteinase 7 (MMP7); matrilysin | 16.9 | 3.0 | 18 |
| plasminogen activator inhibitor, type 2, placental | 3.0 | 12.9 | 429 |
| (PAI-2; PLANH2); monocyte ARG-serpin; | | | |
| urokinase inhibitor | | | |
| plasminogen activator, tissue-type | 11.5 | 3.0 | 26 |
| (T-plasminogen activator; TPA) | | | |
| tenascin (TN); hexabrachion (HXB); cytotactin; | 4.0 | 18.9 | 476 |
| neuronectin; GMEM; miotendinous antigen; | | | |
| glioma-associated extracellular matrix antigen | | | |
| tissue inhibitor of metalloproteinase 1 (TIMP1); | 13.5 | 34.6 | 257 |
| erythroid potentiating activity (EPA); fibroblast | | | |
| collagenase inhibitor | | | |
| 3-hydroxy-3-methylglutaryl-coenzyme A | 3.0 | 14.4 | 481 |
| reductase (HMG-CoA reductase; HMGCR) | | | |
| alkaline phosphatase, type 3, placental | 8.4 | 17.7 | 210 |
| cytochrome P450 reductase | 5.2 | 10.4 | 202 |
| fatty acid synthase (FAS) | 6.3 | 14.0 | 222 |
| lactate dehydrogenase M chain | 10.6 | 22.0 | 206 |
| ornithine decarboxylase (ODC) | 4.1 | 24.6 | 604 |
| S-adenosylmethionine synthetase gamma form | 3.0 | 8.7 | 291 |
| (EC 2.5.1.6); methionine adenosyltransferase; | | | |
| ADOMET synthetase; MAT-II | | | |
| fibroblast growth factor 8 (FGF8); androgen- | 3.3 | 15.3 | 467 |
| induced growth factor (AIGF); HBGF8 | | | |
| Insulin-induced protein 1 | 3.0 | 25.4 | 847 |
| interleukin-1 receptor antagonist protein (IL-1RA; | 5.8 | 19.4 | 335 |
| IRAP) | | | |
| interleukin-6 (IL-6); B-cell stimulatory factor 2 | 3.0 | 29.7 | 992 |
| (BSF2); interferon beta-2 (IFNB2); hybridoma | | | |
| growth factor | | | |
| interleukin-8 (IL-8); monocyte-derived neutrophil | 3.8 | 44.6 | 1171 |
| chemotactic factor (MDNCF); T-cell chemotactic | | | |
| factor; neutrophil-activating protein 1 (NAP1); | | | |
| lymphocyte-derived neutrophil-activating factor | | | |
| (LYNAP); protein 3-10C | | | |
| macrophage inhibitory cytokine 1 (MIC1) | 49.9 | 14.6 | 29 |
| ras homolog gene family member C (RHOC; | 15.3 | 7.6 | 50 |
| ARHC); ARH9; H9 | | | |
| ras-related C3 botulinum toxin substrate 1 | 5.5 | 13.6 | 249 |
| (RAC1); RAS-likeprotein TC25 | | | |
| rho GDP dissociation inihibitor 2 (RHO GDI2; | 12.8 | 3.0 | 23 |
| RHO-GDI beta); LY-GDI; ARHGDIB; GDID4 | | | |
| rho-related GTP-binding protein RhoE; Rho8; | 7.0 | 22.5 | 321 |
| ARHE | | | |
| transcription factor AP-1; c-jun proto-oncogene; | 11.2 | 35.7 | 318 |
| avian sarcoma virus 17 oncogene homolog | | | |
| transcription factor C/EBP alpha (CEBPA); | 3.0 | 6.8 | 228 |
| CCAAT/enhancer protein binding alpha | | | |
| transcription factor NF-kappa-B p100; nuclear | 3.0 | 6.9 | 231 |
| factor NF-kappa-B p100 subunit; nuclear factor | | | |
| NF-kappa-B p52 subunit; H2TF1; oncogene | | | |
| lyt-10 | | | |
| vascular endothelial growth factor (VEGF); | 3.0 | 14.0 | 467 |
| vascular permeability factor (VPF) | | | |
| epidermal fatty acid-binding protein 5 (FABP5; | 3.0 | 9.8 | 327 |
| EFABP); psoriasis-associated fatty acid-binding | | | |
| protein homolog (PAFABP) | | | |
| Gelsolin; actin depolymerizing factor (ADF); | 18.4 | 9.1 | 50 |
| brevin | | | |
| serine palmitoyl transferase | 3.9 | 7.8 | 203 |
| small proline-rich protein 1B (cornifin) | 147.3 | 314.8 | 214 |
| glutathione peroxidase (GSHPX1; GPX1) | 36.8 | 15.5 | 42 |
| HSP70.1; 70-kDa heat shock protein 1 (HSPA1) | 13.9 | 41.1 | 297 |
| HSPA9B; mitochondrial stress-70 protein; 75-kDa | 3.7 | 8.7 | 238 |
| glucose-regulated protein (GRP75); peptide- | | | |
| binding protein 74 (PBP74); mortalin (MOT) | | | |

The methods of the invention thus enable a better comprehension of the syntheses of various molecules during epidermal ageing and can enable the screening of active principles aiming to provide an indication of or to limit the modifications observed during epidermal ageing.

### Example 13

### Test of Effectiveness of a Cosmetic Active Applied Topically onto the Reconstructed Skins Called <<Young and Aged>> Reconstructed Skins

600,000 fibroblasts called <<young>> fibroblasts (extraction from a breast biopsy) and <<aged>> fibroblasts (extraction from a face biopsy obtained after a face-lift) are extracted and amplified until passage 6 (sixth amplification by trypsination) as described in Example 1 and are then sown on dermal substrates based on collagen-glycosaminoglycan-chitosan, in a DMEM-Glutamax culture medium supplemented with 10% of hyclone II calf serum, ascorbic acid-2-phosphate at a final concentration of 1 millimolar, EGF or Epidermal growth factor at a final concentration of 10 ng/mL, penicillin at a final concentration of 100 UI/milliliter, amphotericin B at a final concentration of 1 microgram/milliliter, and gentamycin at a final concentration of 20 micrograms/milliliter, and for a period of culture of 21 days.

Then, 600,000 keratinocytes called <<young>> keratinocytes (extraction from a breast biopsy) and <<aged>> keratinocytes (extraction from a face biopsy obtained after a face-lift) are amplified as described in Example 1 and are then sown in passage 1 (first amplification by trypsination) on the dermal equivalents in a DMEM-Glutamax/Ham F-12 (ratio 3/1 v/v) culture proliferation medium supplemented with 10% of Hyclone II calf serum, ascorbic acid-2-phosphate at a final concentration of 1 millimole/litre, EGF at a final concentration of 10 ng/mL, hydrocortisone at a final concentration of 0.4 micrograms/milliliter, umulin at a final concentration of 0.12 UI/milliliter, isuprel at a final concentration of 0.4 micrograms/milliliter, triiodothyronine at a final concentration of 2.10⁻⁹ molar, adenine at a final concentration of 24.3 micrograms/milliliter, penicillin at a final concentration of 100 UI/milliliter, amphotericin B at a final concentration of 1 microgram/milliliter, and gentamycin at a final concentration of 20 micrograms/milliliter, and for a period of immersion culture of 5 days.

The cultures are then placed at the air-liquid interface for 14 days in a differentiation medium constituted of the culture medium used for the immersion culture, except the calf serum, the hydrocortisone, the isuprel, the triiodothyronine and the umulin.

8 μl of a cosmetic formulation placebo and of a cosmetic formulation containing 3% of active are applied onto the reconstructed skins called <<young and aged reconstructed skins>>. After 2 days of culture in differentiation medium, the cosmetic formulations are removed very delicately from the reconstructed skins and are replaced with the same amount of fresh formulations. This manipulation is renewed 7 times, i.e. 14 additional days of culture. At the end of experimentation, the cosmetic formulations are removed very delicately from the reconstructed skins, the reconstructed skins are rinsed in PBS and then immersed in Tri Reagent® (Sigma). The effectiveness of the treatment with the cosmetic active in formulation is evaluated by the <<cDNA array>> method according to the protocol described in Example 9.

### Example 14

### Test of Effectiveness of a Cosmetic Active Applied Systemically in Models of Reconstructed Dermis Called <<Young and Aged>> Reconstructed Dermis

In Example 12, it is demonstrated that the fibronectin content decreased in models using the cells called aged cells with respect to models using aged cells (decrease of 43%).

Reconstructed dermis are made as described in Example 9. Deliner® (maize extract, Coletica, Lyons) is used at 2% in the culture medium which is changed every two days. The culture media are collected and the fibronectin content is quantified by Dot-Blot as defined in Example 8, followed by an image analysis. The increase in the fibronectin content is of 25%. Deliner® is therefore indeed capable of preventing the decrease in the synthesis of fibronectin which is affected by ageing.

What is claimed is:

1. 1. A method of identifying an eventual modification of at least one biological parameter of living cells which comprises the steps of:
a) using at least one of young living cells and of aged living cells, in a three-dimensional tissue model,
b) performing at least one compared analysis of said aged cells with said young cells, said compared analysis being selected from the group consisting of a compared proteomic profile analysis, a compared transcriptomic profile analysis, and a compared genomic profile analysis; and
c) identifying whether at least one biological parameter of said living cells is eventually modified in said aged cells.

2. The method of claim 1, wherein the young cells and the aged cells are both used in a three-dimensional tissue model.

3. The method of claim 1, wherein said biological parameter, is at least one difference between the metabolism of the young cells and the metabolism of the aged cells.

4. The method of claim 1, wherein the young cells of said step a) are selected from the group consisting of: cells originating from a biopsy from a young donor, cells which have not been multiplied, corresponding to a relatively small number of in vitro passages, and cells taken from a biopsy taken in areas which are not regularly exposed to the sun.

5. The method of claim 4, wherein a young donor is of an age of less than 45 years old.

6. The method of claim 4, wherein said not exposed cells are taken from an area of the body selected from the group consisting of: the body, the breast, the abdomen, and the foreskin.

7. The method of claim 1, wherein the aged cells of said step b) are selected from the group consisting of: cells originating from a biopsy from an aged donor, cells which have undergone a significant number of in vitro passages, and cells which originate from a biopsy taken in an area exposed regularly to the sun.

8. The method of claim 7, wherein an aged donor is of an age of greater than 45 years old.

9. The method of claim 7, wherein said sun exposed cells are taken from an area of the body selected from the group consisting of: the hand, the face, the neck, and the nape.

10. The method of claim 1, wherein the aged cells of said step b) are young cells which have been integrated in a three-dimensional tissue model comprising at least one cell type, artificially aged by a culture prolonged over a long period of time.

11. The method of claim 10, wherein said long period of time is greater than 1 month.

12. The method of claim 1, wherein said young or aged cells are from at least one mammal selected from the group consisting of: a human being, and an animal.

13. The method of claim 1, wherein the proteomic profile analysis is at least one analysis selected from the group consisting of: a bidimensional electrophoresis, a protein array, a cytokine array, and a combined ELISA determination.

14. The method of claim 1, wherein the genomic profile analysis is at least one analysis selected from the group consisting of: a DNA array, a polymerase chain reaction multiplex (PCR-multiplex), a polymerase chain reaction (PCR), and a real time polymerase chain reaction (real time PCR).

15. The method of claim 1, wherein the transcriptomic profile analysis is at least one analysis selected from the group consisting of: an RNA array, a cDNA array, a reverse transcription polymerase chain reaction multiplex (RT-PCR-multiplex), a reverse transcription polymerase chain reaction (RT-PCR), and a real time reverse transcription polymerase chain reaction (real time RT-PCR).

16. The method of claim 1, wherein said tissue model is cultivated under conditions which maintain, at least partially a cell metabolism.

17. The method of claim 1, wherein said tissue model is preserved under conditions which maintain at least partially a cell metabolism.

18. The method of claim 1, wherein said tissue model is cultivated and preserved under conditions which maintain at least partially a cell metabolism.

19. The method of claim 1, wherein said tissue model comprises at least fibroblasts.

20. The method of claim 1, wherein said tissue model comprises at least keratinocytes.

21. The method of claim 1, wherein said living cells are selected from the group consisting of: normal cells, healthy cells, pathological cells, and cells which originate from cell-lines.

22. The method of claim 1, wherein said tissue model is selected from the group of models consisting of: a model of connective matrix, called dermis in the case of skin and called chorion in the case of a mucous membrane, containing mainly stromal cells, an epithelium model constituted mainly of epithelial cells, an epidermis model constituted mainly of keratinocytes, a skin model constituted of an epidermis and of a dermis, and a mucous membrane model constituted of an epithelium and of a chorion.

23. The method of claim 1, wherein said tissue model comprises a matrix support.

24. The method of claim 23, wherein said matrix support is selected from the group consisting of: an inert support containing stromal cells, a gel or a membrane comprising stromal cells, and a porous matrix comprising stromal cells.

25. The method of claim 24, wherein said stromal cells are fibroblasts.

26. The method of claim 24, wherein said semi-permeable synthetic membrane is selected from the group consisting of: a semi-permeable nitrocellulose membrane, a semi-permeable nylon membrane, a teflon membrane, a teflon sponge, a semi-permeable membrane of polycarbonate, a semi-permeable membrane of polyethylene, a semi-permeable membrane of polypropylene, a semi-permeable membrane of polyethylene terephthalate (PET), a semi-permeable Anopore™ inorganic membrane, a cellulose acetate membrane, a cellulose ester (HATF) membrane, a semi-permeable Biopore-CM™ membrane, and a semi-permeable polyester membrane.

27. The method of claim 22, wherein said model of connective matrix comprises a cell culture-treated plastic.

28. The method of claim 24, wherein said gel or membrane is based on at least one component selected from the group consisting of: hyaluronic acid, collagen, fibronectin, fibrin, and mixture thereof.

29. The method of claim 24, wherein said porous matrix is made from a component selected from the group consisting of: collagen containing at least one glycosaminoglycan, collagen containing chitosan, collagen containing at least one glycosaminoglycan and chitosan.

30. The method of claim 22, wherein said tissue model is selected from the group consisting of an epidermis tissue model and an epithelium tissue model, comprising a matrix support which is selected from the group consisting of: an inert support sown beforehand with stromal cells and then with epithelial cells, an inert support not sown beforehand with stromal cells but with epithelial cells, and a film or a membrane sown beforehand with stromal cells and then with epithelial cells.

31. The method of claim 30, wherein said tissue model comprises further added cells in the epithelial part.

32. The method of claim 31, wherein said added cells are selected from the group consisting of: epithelial cells, pigmentary cells, immunocompetent cells, and nerve cells.

33. The method of claim 32, wherein said immunocompetent cells are Langerhans cells.

34. The method of claim 1, wherein said tissue model is selected from the group consisting of: a reconstructed skin model comprising a dermal matrix support, a reconstructed skin model comprising a chorion matrix support, a reconstructed mucous membrane comprising a dermal matrix support, and a reconstructed mucous membrane comprising a chorion matrix support.

35. The method of claim 34, wherein said matrix support is selected from the group consisting of: an inert support containing stromal cells, a gel comprising stromal cells, a porous matrix, and a de-epidermisized dermis or dead dermis.

36. The method of claim 35, wherein said stromal cells are fibroblasts.

37. The method of claim 35, wherein said inert support is a semi-permeable synthetic membrane.

38. The method of claim 37, wherein said semi-permeable synthetic membrane is selected from the group consisting of: a semi-permeable nitrocellulose membrane, a semi-permeable nylon membrane, a teflon membrane, a teflon sponge, a semi-permeable membrane of polycarbonate, a semi-permeable membrane of polyethylene, a semi-permeable membrane of polypropylene, a semi-permeable membrane of polyethylene terephthalate (PET), a semi-permeable Anopore inorganic membrane, a cellulose acetate membrane, a cellulose ester (HATF) membrane, a semi-permeable Biopore-CM membrane, and a semi-permeable polyester membrane.

39. The method of claim 35, wherein said gel is based on at least one component selected from the group consisting of: collagen, hyaluronic acid, fibronectin, fibrin and any mixture thereof.

40. The method of claim 35, wherein said porous matrix is made from at least one component selected from the group consisting of: collagen containing at least one one glycosaminoglycan, collagen containing chitosan, collagen containing at least one glycosaminoglycan and chitosan.

41. The method of claim 35, wherein said dermis is of a source selected from human or animal.

42. The method of claim 35, wherein said matrix support is sown with epithelial cells in order to obtain a reconstructed mucous membrane.

43. The method of claim 35, wherein said matrix support is sown with keratinocytes in order to obtain a reconstructed skin.

44. The method of claim 1, wherein said tissue model used comprises a model in which at least one additional cell type has been incorporated.

45. The method of claim 44, wherein said additional cell type that has been incorporated is at least one type selected from the group consisting of: endothelial cells (EC), immune cells, dendritic cells, adipose cells, and skin appendices.

46. The method of claim 44, wherein said immune cells are selected from the group consisting of: lymphocytes, and macrophages.

47. The method of claim 44, wherein said skin appendices are selected from the group consisting of: body hair, hair, and sebaceous glands.

48. A method for carrying out the screening of at least one potentially active substance reversing at least one biological parameter modified during ageing as defined in claim 1.

49. The method of claim 48, which comprises:
A/ placing said potentially active substance in contact with the aged cells as defined in claim 1, sown in a cell model or a tissue model as defined in claim 1, for a period of time sufficient to enable said potentially active substance to act;
B/ sewing cells as defined in claim 1 in a cell model or tissue model as defined in claim 1;
C/ carrying out at least one analysis selected from the group consisting of: a proteomic analysis, a transcriptomic analysis, and a genomic analysis, for making the study of the action of said substance on the cell metabolism of said aged cells;
D/ comparing the cell metabolism of said aged cells in the presence of the potentially active substance with the metabolism of said aged cells or of the young cells without the presence of said substance, and;
E/ identifying the presence or the absence of activity of said potentially active substance, in order to provide an indication of the modification of the biological parameter identified as being modified during ageing.

50. The method of claim 49, wherein said identification of the presence or absence of activity of said potentially active substance in step E/ comprises identifying a positive or negative effect of said substance.

51. A method of identifying at least one potentially active substance reversing at least one biological parameter modified during ageing comprising:
a) culturing young cells, which are used as reference;
b) culturing aged cells having a biological parameter modified with respect to the young cells, in the presence of at least one potentially active substance, for a period of time sufficient to enable said potentially active substance to potentially act on the cell metabolism of said cells, so as to substantially recover the metabolism of the young cells;
c) performing at least one analysis selected from the group consisting of: a proteomic analysis, a transcriptomic analysis, and a genomic analysis, of the aged cells cultivated in the presence or not of an potentially active substance; and
d) comparing the analysis carried out in c) with the proteomic analysis and/or transcriptomic analysis, and/or genomic analysis, of young living cells cultivated without the presence of said potentially active substance, as described in a).

52. The method of claim 51, wherein the method comprises after the comparison of the analyses carried out in c) and d):
e) eventually identifying at least one active substance reversing at least one biological parameter which is modified by the ageing.

53. The method of claim 51, wherein in step a), said young cells are selected from the group consisting of: cells originating from a biopsy from a young donor, cells which have not been multiplied very much, corresponding to a relatively small number of in vitro passages, and cells taken from a biopsy not exposed very much to solar radiation.

54. The method of claim 51, wherein in step b) said aged cells are selected from the group consisting of cells originating from a biopsy from an aged donor, cells which have undergone a significant number of in vitro passages, and cells which originate from a biopsy taken in an area which is exposed to the sun.

55. A method of identifying at least one potentially active substance capable of providing an indication of the modification of at least one biological parameter which is modified during ageing comprising:
a) placing said potentially active substance in contact with the aged cells as defined in claim 7 sown in a tissue model as defined in claim 22, for a period of time sufficient to enable said potentially active substance to act;
b) performing at least one analysis selected from the group consisting of: a proteomic analysis, a transcriptomic analysis, and a genomic analysis, of the aged cells placed in contact with these substances; and
c) comparing the analysis carried out in b) with the proteomic analysis and/or transcriptomic analysis and/or genomic analysis, of living cells which are cultivated without the-presence of said potentially active substance.

56. The method of claim 55, wherein the method comprises a further step d) following the comparison of the analysis carried out in c): identifying at least one active substance capable of providing an indication of the modification of at least one biological parameter which is modified by the ageing.

57. A substance active in the field of cosmetics selected by a method defined in claim 48.

58. A substance active in the field of pharmacy selected by a method defined in claim 48.

59. An active substance providing at least one effect selected from the group consisting of: an effect of reversing a biological parameter which is identified as being modified during ageing, and an effect of providing an indication of the modification thereof, this parameter being identified by making compared studies between cell models using young cells and cell models using aged cells, one at least of these models being a tissue model comprising at least either fibroblasts or keratinocytes.

61. A cosmetic composition comprising a substance of claim 57.

62. A pharmaceutic composition comprising a substance of claim 58.
